(19) United States
(12) Patent Application Publication
(10) Pub. No.: US 2023/0293339 A1
(43) Pub. Date: Sep. 21, 2023

(54) Absorbent Hygienic Article Having an Integrated Drainage Mechanism

(71) Applicant: Gary Bruce James, II, North Hollywood, CA (US)

(72) Inventor: Gary Bruce James, II, North Hollywood, CA (US)

(21) Appl. No.: 17/700,328

(22) Filed: Mar. 21, 2022

(51) Int. Cl. A61F 5/455; A61F 13/45

(52) U.S. Cl. CPC A61F 5/455; A61F 13/45; A61F 2013/4506

(57) ABSTRACT

The present disclosure provides a hygienic article comprising a multi-layered absorbent pad having a built-in drainage mechanism in the form of a plastic tube that is affixed between two layers of the pad. The portion of the tubing that rests in the pad has a plurality of inlets for capturing excess fluid that accumulates between the layers, funnelling it into the tubing. The other end of the tubing extends out and ends in a connector element that allows it to form a watertight seal with a suction apparatus such as an aspirator. Once the pad is soiled the pad may simply be exchanged for another one which is affixed to the end of the tubing with the fluid inlets.

### FIELD OF INVENTION

The present invention relates generally to an absorbent article having an integrated drainage mechanism. More specifically, the present invention relates to a hygienic pad for incontinence configured to prevent oversaturation of fluid by incorporation of an outflow pipe.

### BACKGROUND

Many individuals with serious health difficulties suffer from incontinence of varying levels of severity, from the inability to properly control their bladder to complete incontinence. Such individuals are assigned careers, often their loved ones and family, to assist them with this. However, it is impossible for a career to perfectly predict when episodes of incontinence will occur, and it is therefore a common situation that the individual is left sitting in their own urine or excrement. Not only is this unpleasant, but it can lead to disorders of and deterioration of the skin due to the exposure to high levels of moisture for prolonged periods, which in turn can become infected.

Sanitary items and other solutions do exist for mitigating the effects of incontinence, usually consisting of an absorbent pad article, however there are none available on the market capable of effectively absorbing the sheer volumes of fluid produced by the human body. A single instance of incontinence will usually saturate the absorbent pad and leave the individual's skin in contact with the excess moisture.

It is within this context that the present invention is provided.

### SUMMARY

The present disclosure provides a hygienic article comprising a multi-layered absorbent pad having a built-in drainage mechanism in the form of a plastic tube that is affixed between two layers of the pad. The portion of the tubing that rests in the pad has a plurality of inlets for capturing excess fluid that accumulates between the layers, funnelling it into the tubing. The other end of the tubing extends out and ends in a connector element that allows it to form a watertight seal with a suction apparatus such as an aspirator. Once the pad is soiled the pad may simply be exchanged for another one which is affixed to the end of the tubing with the fluid inlets.

Thus, according to one aspect of the present disclosure there is provided a hygienic article comprising: a multi-layered absorbent pad, an outer layer of the pad having a first surface adapted to be placed against the human body; and a length of flexible tubing having a first end comprising a connector element being adapted to form a sealed connection with a suction drainage apparatus, the tubing being divided into a first portion and a second portion, the first portion of the tubing spanning from the first end to the second portion, and the second portion spanning from the first portion to an opposing second end, the second portion having formed therein a plurality of inlets; wherein the entirety of the second portion of the tubing rests between two layers of the absorbent pad and is secured thereto by one or more coupling mechanisms.

In some embodiments, the absorbent pad is elongated in shape with first and second longitudinal edges running along its length. In such embodiments, the one or more coupling mechanisms include stitching at a first point at one end of the elongated pad and stitching at a second point at the opposing end of the elongated pad.

In some embodiments, inlets of the second portion of the tubing are formed of one or more openings disposed at different points about the circumference of the tubing.

In some embodiments, the tubing is formed of Poly-Vinyl-Chloride, PVC.

In some embodiments, a second outer layer of the absorbent pad opposite the first outer layer has a greater surface area than the other layers of the pad, thereby providing an outer lip area for folding about clothing articles or catching dripped fluids.

In some embodiments, the first portion of the tubing that extends out from the absorbent pad is at least one metre in length.

In some embodiments, the pad is a disposable pad and the coupling between the tubing and the absorbent pad is detachable, allowing fresh pads to be exchanged for soiled ones easily.

In some embodiments, the connector element is a female connector for coupling to a suction aspirator machine.

In some embodiments, the absorbent pad is coupled to or integrated with an article of clothing such as an undergarment.

### BRIEF DESCRIPTION OF THE DRAWINGS

Various embodiments of the invention are disclosed in the following detailed description and accompanying drawings.

FIG. 1 illustrates an isometric view of an example configuration of the hygienic article of the present disclosure.

FIG. 2 illustrates an exploded view of the example configuration of the hygienic article with the layers of the absorbent pad separated and the portion of the end of the tubing with the fluid inlets visible between.

FIG. 3 illustrates the flexible tubing of the example configuration with the portion having the fluid inlets having been removed from the absorbent pad.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

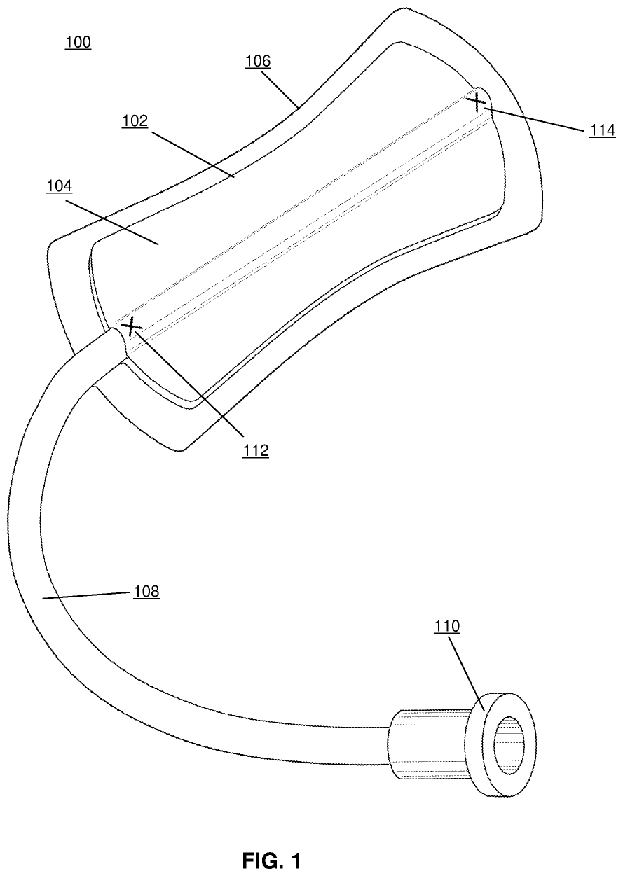

Referring FIG. 1, an isometric view of an example configuration of the hygienic article 100 of the present disclosure is shown in an assembled state.

As can be seen, the article 100 is comprised of a multi-layered absorbent pad 102 having a first surface 104 that is configured to lie against the skin of the human body. This surface may comprise extra padding and be formed of soft material. An opposing outer layer 106 is adapted to face outwards towards the wearer's garments. In the present example configuration, this outer layer 106 has a larger surface area than the other layers, forming a lip that extends out further and can be used to either wrap around a wearer's garments or catch excess fluid.

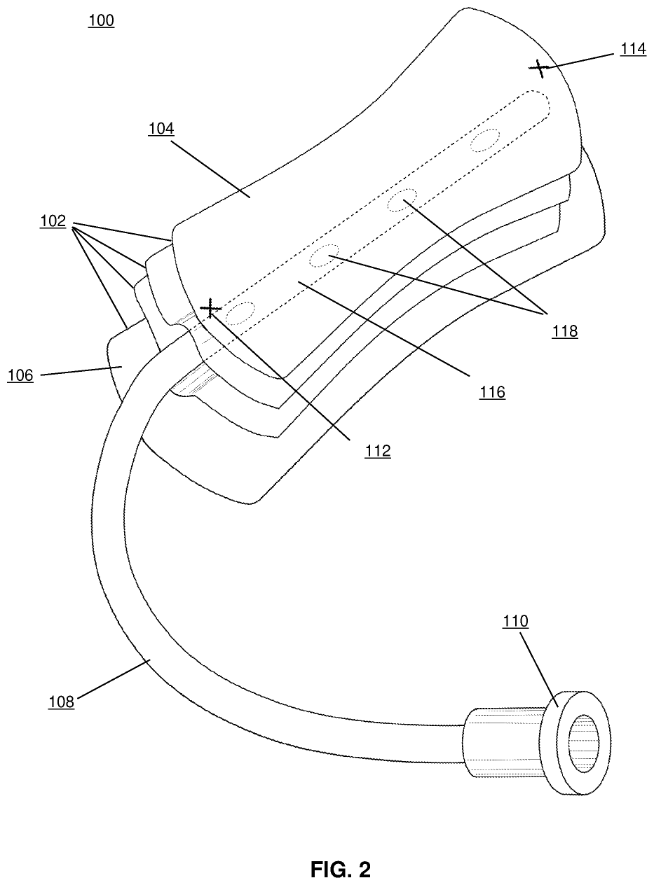

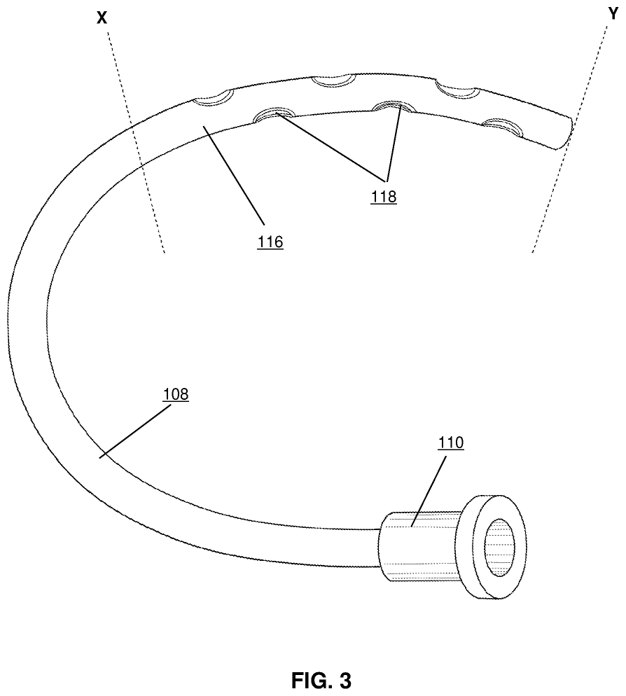

A flexible plastic tube 108 rests between two central layers of absorbent pad 102, the portion sandwiched between them having a plurality of fluid inlets (visible in FIG. 2 and FIG. 3) for draining excess fluid that accumulates within the pad to prevent oversaturation. The exposed portion of the tubing is sealed, and ends in a connector element 110. In the present example this is a female connector for forming a watertight seal with a standard machine suction aspirator that will encourage the excess fluid accumulated in the pad to flow into the fluid inlets.

The portion of the tubing that rests within the pad 102 can be affixed in place within the pad by any suitable means. In the present example the tubing is stitched to the pad at a first position 112 and a second position 114 at opposing ends of the elongated shape.

It is noted that the illustrations provided herein are meant to clarify the working of the article and are not necessarily to scale. In some examples the portion of the tubing extending form the pad may be a metre long or more to facilitate connecting to an aspirator machine without overly restricting the movements of the wearer.

The tubing 108 can be formed of any suitable flexible material that can form a watertight seal. Plastics such as polyvinyl chloride, PVC, are preferred for their structural integrity and flexibility. Having the tubing be at least partially flexible allows the entire article to mould to the shape of the wearer's body.

Referring to FIG. 2, an exploded view of the example configuration of the hygienic article 100 is shown with the layers 102 of the absorbent pad separated and the portion of the end of the tubing 116 with the fluid inlets 118 visible between (shown by the dashed lines).

Referring to FIG. 3, the flexible tubing 108 is shown on its own, with the portion 116 (which spans from line X to line Y) with the fluid inlets 118 disposed thereon having been removed from the absorbent pad. As can be seen, in the present example the fluid inlets 118 are simply in the form of holes in the plastic tubing, however valve-controlled inlets may in some cases be beneficial for certain types of suction apparatus.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the hygiene article have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A hygienic article comprising:
a multi-layered absorbent pad, an outer layer of the pad having a first surface adapted to be placed against the human body; and
a length of flexible tubing having a first end comprising a connector element being adapted to form a sealed connection with a suction drainage apparatus, the tubing being divided into a first portion and a second portion, the first portion of the tubing spanning from the first end to the second portion, and the second portion spanning from the first portion to an opposing second end, the second portion having formed therein a plurality of inlets;
wherein the entirety of the second portion of the tubing rests between two layers of the absorbent pad and is secured thereto by one or more coupling mechanisms.

2. A hygienic article according to claim 1, wherein the absorbent pad is elongated in shape with first and second longitudinal edges running along its length.

3. A hygienic article according to claim 2, wherein the one or more coupling mechanisms include stitching at a first point at one end of the elongated pad and stitching at a second point at the opposing end of the elongated pad.

4. A hygienic article according to claim 1, wherein inlets of the second portion of the tubing are formed of one or more openings disposed at different points about the circumference of the tubing.

5. A hygienic article according to claim 1, wherein the tubing is formed of Poly-Vinyl-Chloride, PVC.

6. A hygienic article according to claim 1, wherein a second outer layer of the absorbent pad opposite the first outer layer has a greater surface area than the other layers of the pad, thereby providing an outer lip area for folding about clothing articles or catching dripped fluids.

7. A hygienic article according to claim 1, wherein the first portion of the tubing that extends out from the absorbent pad is at least one metre in length.

8. A hygienic article according to claim 1, wherein the pad is a disposable pad and the coupling between the tubing and the absorbent pad is detachable, allowing fresh pads to be exchanged for soiled ones easily.

9. A hygienic article according to claim 1, wherein the connector element is a female connector for coupling to a suction aspirator machine.

10. A hygienic article according to claim 1, wherein the absorbent pad is coupled to or integrated with an article of clothing such as an undergarment.
